(19) United States
(12) Patent Application Publication
(10) Pub. No.: US 2019/0282591 A1
(43) Pub. Date: Sep. 19, 2019

(54) FINASTERIDE AND SILDENAFIL COMPOSITIONS AND APPLICATIONS

(71) Applicant: BLUE GOOSE DRUGS INC., SOUTH LANCASTER (CA)

(72) Inventors: ANDREW HANNA, CORNWALL (CA); JONATHAN HUZA, SOUTH LANCASTER (CA)

(21) Appl. No.: 16/356,314

(22) Filed: Mar. 18, 2019

Related U.S. Application Data
(60) Provisional application No. 62/643,893, filed on Mar. 16, 2018.

(51) Int. Cl. A61K 31/58; A61K 31/519; A61K 31/506; A61K 9/00

(52) U.S. Cl. CPC A61K 31/58; A61K 31/519; A61K 9/0014; A61K 9/0056; A61K 31/506

(57) ABSTRACT

The human body is mediated by a large number of chemicals and chemical processes where imbalances can result in an abnormal condition that affects part or all of the human body. Amongst these conditions are hair loss and male impotence or erectile dysfunction, both of which can have psychological consequences for the patient and others as they can be tied to relationship difficulties and self-image. 5α-reductase Type II inhibitors prevent DHT production and reduce androgen activity in key tissues such as prostate and scalp. Similarly, an inhibitor of cyclic guanosine monophosphate (cGMP) specific phosphodiesterase type 5 (PDE5) can lead to improved vasodilation and blood flow. It would be beneficial to provide patients with either of these treatments within a topical cream form allowing localized targeted delivery.

### CROSS-REFERENCE TO RELATED APPLICATION

This application claims the benefit of priority from U.S. Provisional patent application 62/643,893 filed Mar. 16, 2018 entitled “Finasteride and Sildenafil Compositions and Applications”, the entire contents of which are incorporated herein by reference.

### FIELD OF THE INVENTION

This invention relates generally relates to medicaments and other agents and more specifically, the present invention relates to the delivery of medicaments or other agents such as the 5α-reductase inhibitor (5α, 17β)-N-(1,1-di methylethyl)-3-oxo-4-azaandrost-1-ene-17-carboxamide (finasteride) within a topical lotion, cream, foam, ointment gel or shampoo and an inhibitor of cyclic guanosine monophosphate (cGMP) specific phosphodiesterase type 5 (PDE5) such as sildenafil citrate (C₂₈H₃₈N₆O₁₁S) within a sublingual or buccal mucosal format.

### BACKGROUND OF THE INVENTION

The human body is an extremely complex interlinked system mediated by a large number of chemicals and chemical processes. Accordingly, any imbalance of any one or more of these chemicals or chemical processes can result in an abnormal condition that affects part or all of the human body. Typically referred to as a “disease” or a “condition” in order to differentiate imbalances caused by an external force, an “injury”, then many diseases arise from an external infection or pathogen whilst others arise from biological degradations triggered from one or more factors of which age, genetics, and weight are the most common. A disease typically consists of a disorder of a structure or function. Over history a wide range of medicines have been used to address specific diseases although with the advancements in the 19^{th} and 20^{th} centuries in respect of diagnostic techniques etc. the number of identified diseases, their causes, and their medicinal treatments has increased substantially. With the concurrent developments in chemistry a wide range of artificial drugs have been added to the physician's armoury to address specific diseases by targeting the specific chemical imbalance(s) and/or chemical process(es).

Amongst these multitude of diseases and conditions are hair loss, which impacts both men and women, and male impotence. However, with treatments providing the requisite pharmaceutical in the requisite dosage and an acceptable format additional issues affect their use including, but not limited to, the safety of children and pets as well as, according to the pharmaceutical/dosage, women and/or men.

A variety of conditions lead to hair loss and although the effect is primarily cosmetic, there is an adverse psychological impact on the affected patients. Because of this negative impact on body image, society expends substantial financial resources for various pharmacological agents, cosmetic treatments, surgical procedures, and prosthetic articles to counteract hair loss. Current pharmacological treatments for hair loss include the user of minoxidil and finasteride. Minoxidil appears to lengthen the duration of the anagen stage of hair growth by increasing the blood supply to the follicle but appears to have to no direct effect in stimulating hair follicle development or growth. Topical treatment with the drug must be carried out continuously because cessation of treatment results in a more severe hair loss pattern as the blood supply to the follicle decreases. In contrast, finasteride is a 5α-reductase Type II inhibitor targeting the intracellular enzyme responsible for converting androgen testosterone into dihydrotestosterone. By inhibiting 5α-reductase and thus preventing DHT production, finasteride reduces androgen activity in tissues like the prostate gland and the scalp. The drug is beneficial for patients with androgenetic alopecia because the condition is associated with elevated levels of dihydrotestosterone, which is believed to shorten the anagen stage of the hair follicle development.

Today, minoxidil is applied via a topical foam and finasteride is taken orally in tablet form. However, finasteride due to its absorption throughout the body and degradation during ingestion can result in a range of side effects such as ambiguous genitalia in developing male fetuses currently limiting its use to men. Like Minoxidil, treatment with finasteride must be continuous because cessation of treatment leads to gradual progression of the disorder. Accordingly, it would be beneficial to find an alternative treatment that increases absorption within the targeted area of the human body, e.g. the scalp. Accordingly, the inventors have established that a topical treatment can result in subcutaneous or intradermal administration to induce development of hair follicles and promote hair growth.

Male impotence, also referred to as erectile dysfunction (ED), is a type of sexual dysfunction characterized by the inability to develop or maintain an erection of the penis during sexual activity. Erectile dysfunction can have psychological consequences as it can be tied to relationship difficulties and self-image. Sildenafil protects cyclic guanosine monophosphate (cGMP) from degradation by cGMP-specific phosphodiesterase type 5 (PDE5) in the corpus cavernosum. Nitric oxide (NO) in the corpus cavernosum of the penis binds to guanylate cyclase receptors, which results in increased levels of cGMP, leading to smooth muscle relaxation (vasodilation) of the intimal cushions of the helicine arteries. This smooth muscle relaxation leads to vasodilation and increased inflow of blood into the spongy tissue of the penis, causing an erection. It has also been found to help patients with premature ejaculation.

Accordingly, sildenafil is a potent and selective inhibitor of cGMP-specific phosphodiesterase type 5 (PDE5) and has a molecular structure similar to that of cGMP and acts as a competitive binding agent of PDE5 in the corpus cavernosum, resulting in more cGMP and better erections. Without sexual stimulation, and therefore lack of activation of the NO/cGMP system, sildenafil itself should not cause an erection. Sildenafil is marketed by Pfizer under the trademark Viagra and under the trademark Revatio for pulmonary hypertension using sildenafil citrate. Other drugs that operate by the same mechanism include tadalafil (marketed under the trademark Cialis by Eli Lilly) and vardenafil (marketed under the trademark Levitra by Bayer AG, GlaxoSmithKline, and Schering-Plough).

Current dosage regimens for Viagra are for adults an initial dose of 50 mg orally once a day, as needed, with a subsequent maintenance dose of 25 mg to 100 mg orally once a day whilst for geriatrics a dose of 25 mg orally once a day is common. However, higher doses may be employed and at doses of 150 mg or more the need for PPP (commonly referred to as “Triple P” and comprising prostaglandin E1, papaverine, and phentolamine) which is provided by intracavernous injection therapy to help with blood flow and develop an erection is not need. As PPP is an injection the risks of developing penial infections is increased.

However, it would be beneficial to provide an alternative delivery mechanism to the current tablets and accordingly the inventors have established a formulation supporting a rapidly dissolving tablet.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate limitations in the prior art relating to medicaments and other agents and more specifically, the present invention relates to the delivery of medicaments or other agents such the 5α-reductase inhibitor (5α,17β)-N-(1,1-dimethylethyl)-3-oxo-4-azaandrost-1-ene-17-carboxamide (finasteride) within a topical lotion, cream, foam, ointment gel or shampoo and an inhibitor of cyclic guanosine monophosphate (cGMP) specific phosphodiesterase type 5 (PDE5) such as sildenafil citrate (C₂₈H₃₃N₆O₁₁S) within a sublingual or buccal mucosal format.

In accordance with an embodiment of the invention there is provided a pharmaceutical composition comprising a 5α-reductase inhibitor and a topical carrier for the 5α-reductase inhibitor.

In accordance with an embodiment of the invention there is provided a pharmaceutical composition comprising an antihypertensive potassium channel opener and a topical carrier for the antihypertensive potassium channel opener.

In accordance with an embodiment of the invention there is provided a pharmaceutical composition comprising:
an inhibitor of cyclic guanosine monophosphate (cGMP) specific phosphodiesterase type 5 (PDE5); and
a non-pharmaceutical carrier allowing the cGMP inhibitor to be in tablet form for placement by a user into their mouth and providing for rapid dissolving allowing absorption of the cGMP inhibitor by the user to be primarily through their mouth.

In accordance with an embodiment of the invention there is provided a pharmaceutical composition comprising:
an inhibitor of cyclic guanosine monophosphate (cGMP) specific phosphodiesterase type 5 (PDE5); and
a non-pharmaceutical carrier allowing the cGMP inhibitor to be in a form for placement by a user into their mouth and providing for absorption of the cGMP inhibitor by the user to be primarily through their mouth; wherein
the non-pharmaceutical carrier provides for the form to be one of a rapidly dissolving tablet; a rapidly dissolving hard candy; a rapidly dissolving lollipop; a chewable tablet; and a jelly candy.

Other aspects and features of the present invention will become apparent to those ordinarily skilled in the art upon review of the following description of specific embodiments of the invention in conjunction with the accompanying figures.

### DETAILED DESCRIPTION

The present invention is directed to medicaments and other agents and more specifically, the present invention relates to the delivery of medicaments or other agents such as the 5α-reductase inhibitor (5α, 17β)-N-(1,1-dimethylethyl)-3-oxo-4-azaandrost-1-ene-17-carboxamide (finasteride) within a topical lotion, cream, ointment gel or shampoo and an inhibitor of cyclic guanosine monophosphate (cGMP) specific phosphodiesterase type 5 (PDE5) such as sildenafil citrate (C₂₈H₃₈N₆O₁₁S) within a sublingual or buccal mucosal format.

The ensuing description provides exemplary embodiment(s) only, and is not intended to limit the scope, applicability or configuration of the disclosure. Rather, the ensuing description of the exemplary embodiment(s) will provide those skilled in the art with an enabling description for implementing an exemplary embodiment. It being understood that various changes may be made in the function and arrangement of elements without departing from the spirit and scope as set forth in the appended claims.

“Finasteride” as used herein and throughout the disclosure refers to the 5α-reductase inhibitor having the molecular formula C₂₃H₃₆N₂O₂ identified by the United States Adopted Names (USAN) Council and the United States Pharmacopeia (USP) Dictionary of Drug Names (1991, p. 257) as N-t-butyl-3-oxo-4-aza-5α-androst-1-ene-17β-carboxamide with the Chemical Abstracts name (5α,17β)-N-(1,1-dimethylethyl)-3-oxo-4-azaandrost-1-ene-17-carboxamide and International Union of Pure and Applied Chemistry (IUPAC) name (1S,3aS,3bS,5aR,9aR,9bS,11aS)-N-tert-butyl-9a, 11a-dimethyl-7-oxo-1,2,3,3a,3b,4,5,5a,6,9b, 10,11-dodecahydroindeno[5,4-f]quinoline-1-carboxamide. Finasteride is a synthetic androstane steroid and 4-azasteroid and behaves as an analogue of androgen steroid hormones like testosterone and dihydrotestosterone (DHT, also known as 5α-dihydrotestosterone (5α-DHT)).

1 “Sildenafil” or “sildenafil citrate” as used herein and throughout the disclosure refers to the chemical having molecular formula C₂₈H₃₃N₆O₁₁S which acts as a phosphodiesterase type-5 inhibitor, vasodilator, and urological agent. Sildenafil citrate is identified by the IUPAC name 5-[2-ethoxy-5-(4-methylpiperazin-1-yl)sulfonylphenyl]-1-methyl-3-propyl-4H-pyrazolo[4,3-d]pyrimidin-7-one; 2-hydroxypropane-1,2,3-tricarboxylic acid.

The terms “drug” or “active agent” are used interchangeably herein and throughout the disclosure to describe a pharmacologically active substance that is present in the composition in a quantity that is sufficient to elicit an intended pharmacological response.

A “pharmaceutical composition” as used herein and throughout the disclosure refers to compositions comprising one or more drugs together with one or more pharmaceutically acceptable excipients as required to prepare a dosage form for the effective delivery of the active agent. Such pharmaceutical compositions can be in the form of solutions, ointments, creams, gels, lotions, suspensions, sprays, foams, microspheres, microemulsions, nanoemulsions, nanoparticles, nanosuspensions, dermal sticks, roll-ons, pumps, patches, tapes, and the like. Certain pharmaceutical compositions of the present invention may be in the form of “permeation enhanced” compositions, or “penetration enhanced” compositions, or “depot” compositions.

A “topical composition” or “topical” composition as used herein and throughout the disclosure refers to a composition that is applied onto the skin surface. Such a “topical” composition may act “locally” or “transdermally”. A “transdermal” composition refers to a composition that can be applied to the body surface, which then may permeate through the stratum corneum or scalp to form a reservoir just beneath the skin surface or may be absorbed systemically to provide desirable drug levels in the circulation.

“Skin depot” or “depot” as used herein and throughout the disclosure refers to a pharmaceutical composition that provides storage of drug within the skin and releases the contained drug to surrounding tissue over a prolonged period of time, and/or delayed to commence after a period of time.

The “scalp” as used herein and throughout the disclosure refers the skin covering the head and is bordered by the face anteriorly and the neck to the sides and posteriorly.

A “rapidly dissolving tablet” (RDT) as used herein and throughout the disclosure refers to tablets that are distinguished from conventional sublingual tablets, lozenges, and buccal tablets which require more than a minute to dissolve in the mouth. RDT's are also known as orally disintegrating tablets (ODTs), orodisperse, mouth-dissolving, quick-dissolve, and fast-melt. RDTs may therefore release the drug or drugs in the mouth for absorption through local oromucosal tissues and through pregastric (e.g., oral cavity, pharynx, and esophagus), gastric (i.e., stomach), and postgastric (e.g., small and large intestines) segments of the gastrointestinal tract (GIT).

“Medi-RDT Base” (base) as used herein and throughout the disclosure refers to a finely granulated powder, typically sugar free, that is compatible with a wide range of active ingredients (drugs).

“Foamil” as used herein and throughout this disclosure refers to a foam which supports the compounding of active ingredient(s) without requiring heating and filtration steps as marketed those by Medisca under the trademark Foamil. Its composition makes it suitable for compounding other active ingredients that are appropriate for use in hair loss applications. The foam has a typical pH value above 3.5 and an alcohol concentration typically below 10% allowing it to be mild and non-irritating to the scalp, thus making it ideal for compounding leave-on treatments to prolong the life cycle of hair.

“Tablet” as used herein and throughout the disclosure refers to a solid composition made by compressing and/or molding a mixture of compositions in a form convenient for swallowing or application to a body cavity.

A “formulation” as used herein and throughout the disclosure refers to refers to any mixture of compositions used to make either a lotion according to embodiments of the invention or tablets according to embodiments of the invention.

A “body cavity” as used herein and throughout the disclosure refers to any body cavity capable of receiving a tablet, including the oral, buccal, sublingual, eye, ear, vaginal, nasal, rectal, and urethral cavities.

An “organic solvent” and “organic solvent residue” as used herein and throughout the disclosure refers to an organic substance that dissolves a solute (a chemically distinct material), resulting in a solution. Such organic solvents may include, but not be limited to, an alcohol, isopropyl alcohol, ethanol, methanol, methylene chloride, acetone, and the like.

A “water soluble polymer” as used herein and throughout the disclosure refers to a polymeric composition, soluble in an aqueous solution.

“Polyvinylpyrrolidone” or “PVP” refers to any of the polymers of vinylpyrrolidone, or derivatives thereof. While PVP is typically made via a free radical polymerization process, any soluble grade polymer of vinylpyrrolidone may be employed within embodiments of the invention where appropriate. Typically, linear PVP polymers are water soluble and cross-linked PVP polymers are not water soluble.

A “saccharide” as used herein and throughout the disclosure refers to any monosaccharide or polysaccharide, or derivative thereof, from any natural or synthetic sources. A saccharide may include, but not be limited to, mannitol, lactose, glucose, sucrose, xylitol, maltose, maltitol, sorbitol, oligosaccharides, and other similar saccharides.

A “pharmaceutically active ingredient” as used herein and throughout the disclosure refers to any medicament, nutritional, palliative, drug or pharmaceutical added to a tablet or lotion as appropriate to the embodiment of the invention.

A “disintegrant” as used herein and throughout the disclosure refers to any composition which decreases the disintegration time (accelerates the rate of disintegration) of a tablet.

A “flavoring” as used herein and throughout the disclosure refers to any composition which adds flavour to or masks the bad taste of a formulation.

An “artificial sweetener” as used herein and throughout the disclosure refers to any synthetic composition that sweetens the taste of a formulation.

A “perfume” as used herein and throughout the disclosure refers to any composition that contributes to the odor or taste, or masks an unpleasant smell, of a formulation.

A “colorant” as used herein and throughout the disclosure refers to any composition that adds color to a formulation.

“Granulating” as used herein and throughout the disclosure refers to the process of blending and mixing a formulation.

“Compressing” as used herein and throughout the disclosure refers to the process of applying compressive force to a formulation, as within a die, to form a tablet.

“Humidifying” and “humidification” as used herein and throughout the disclosure refer to the process of adding moisture to a tablet, as reacting the tablet with a relatively humid (water saturated) environment. The term “relative humidity” is used in its common context and refers to the percentage of water saturation in a gas.

“Drying” and “dried” as used herein and throughout the disclosure refer to a process which decreases the water content of a composition, as the drying of a humidified tablet. A “dried tablet” as used herein and throughout the disclosure refers to a tablet that has been treated in any manner to decrease the amount of water in the formulation, as when a tablet is dried after its initial granulation and compression into a tablet form.

“Filler” as used herein and throughout the disclosure refers to any inert material or composition added to a formulation to add bulk to a formulation.

“Press molding” as used herein and throughout the disclosure refers to any apparatus which places compressive force on a formulation to compress and shape the composition, as with the compression of a wet or dry formulation to create a tablet.

“Physiologically acceptable” as used herein and throughout the disclosure refers to any combination of materials or compositions that are not harmful, i.e., non-toxic, to cells and tissues under physiologic (in vivo) conditions.

A “lollipop” as used herein and throughout the disclosure refers to product exploiting a hard or soft candy on a stick as delivery format for a pharmaceutical compound or composition such that sucking and/or licking of the lollipop releases the pharmaceutical compound or composition into the buccal mucosa of a patient.

A “soft candy” or “jelly candy” (also known as a gummi or gummy) as used herein and throughout the disclosure refers to product exploiting a chewable sweet or candy as delivery format for a pharmaceutical compound or composition such that sucking and/or chewing the soft candy releases the pharmaceutical compound or composition into the buccal mucosa of a patient.

Topical Finasteride Composition

Finasteride is a 5α-reductase inhibitor, specifically the type II and III isoenzymes although the tissues in which the isoforms of 5α-reductase are expressed is unclear since different investigators have obtained varying results with different reagents, methods, and tissues examined. However, common to many studies the isoforms appear to widely expressed. By inhibiting 5α-reductase, finasteride prevents the conversion of testosterone to dihydrotestosterone (DHT) by the type II and III isoenzymes, resulting in a decrease in serum DHT levels by about 65 to 70% and in prostate DHT levels by up to 85 to 90%, where expression of the type II isoenzyme predominates. Unlike triple inhibitors of all three isoenzymes of 5α-reductase like dutasteride which can reduce DHT levels in the entire body by more than 99%, finasteride does not completely suppress DHT production because it lacks significant inhibitory effects on the 5α-reductase type I isoenzyme, with more than 100-fold less inhibitory potency for type I as compared to type II. In addition to inhibiting 5α-reductase, finasteride has also been found to competitively inhibit 5β-reductase (AKR1D1), although its affinity for the enzyme is substantially less than for 5α-reductase (an order of magnitude less than 5α-reductase type I). By inhibiting 5α-reductase and thus preventing DHT production, finasteride reduces androgen activity in tissues like the prostate gland and the scalp.

Male Pattern Hair Loss (MPHL) is a type of “androgenetic alopecia” which is a genetically-mediated disorder that occurs in approximately 50% of men by the age of 50 years. In women, the histological features of the condition are the same as in men, but susceptibility, age at onset, rate of progression and pattern of hair loss differ between genders. After puberty, males begin to lose the scalp hair over the vertex, crown and frontal/parietal areas in a relatively characteristic pattern that is a continuum, typically defined by the Hamilton Norwood scale. The process of hair loss occurs at the level of the hair follicles by “miniaturization” through which the hair follicle becomes progressively smaller both in depth and circumference, and the hair shaft produced becomes shorter and thinner. The ratio of terminal-to-vellus-like hairs may be reduced from an initial approximately 7:1 to less than 2:1. Miniaturization results in increased proportions of club hair shafts or vellus hair shafts. The loss of scalp hair in men is known to be a process driven by the androgen dihydrotestosterone (DHT), which can be inhibited and to some extent reversed by finasteride, which inhibits 5-alpha-reductase II (which converts testosterone to DHT).

Female Pattern Hair Loss (FPHL). In addition to the progression of MPHL, both males and females develop diffuse hair loss in the frontal/parietal scalp called “thinning,” which begins between 12 and 40 years of age. Perhaps more than males, females notice (and complain of) diffuse hair thinning progressively in middle age more than males, perhaps because diffuse alopecia is more noticeable and problematic for females because they do not suffer from MPHL and retain the frontal hairline. In females, thinning is known as “Female Pattern Hair Loss (FPHL)” and may be caused or exacerbated by androgens. Mechanistically, FPHL is thought to share some features with MPHL in terms of progressive reduction in the duration of anagen and progressive follicular miniaturization. As with MPHL, thinning of the hair, especially on the top of head, in addition to affecting younger individuals, can also occur in older individuals when amounts of testosterone and DHT in the body are decreasing. This can either be an extension of FPHL from the earlier years or even start in the latter decades of life (i.e. age-related hair thinning).

Chemical treatments involve the use of drugs for the treatment of certain MPHL. These include, for example, minoxidil (marketed under the trademark Rogaine), which is an antihypertensive drug that opens potassium (K+) channels; and antiandrogens such as finasteride, dutasteride or ketoconazole. Minoxidil and antiandrogens are reasonably effective in stimulating the growth of vellus and miniaturized hair in certain MPHL conditions. Importantly, both minoxidil and finasteride are effective only for as long as it is taken; the hair gained or maintained is lost within 6-12 months of ceasing therapy. Thus, minoxidil and finasteride require continuous treatment for lasting effects. In addition, patients with advanced MPHL may express dissatisfaction with even statistically significant, but cosmetically insignificant increase in hair counts and such frustration may contribute to poor compliance and further unsatisfactory outcomes. Further, minoxidil use is further complicated by the fact that it is messy and can leave a residue.

Finasteride is an inhibitor of the steroid 5-α-reductase, for use in the compositions and methods of the present invention, is a synthetic 4-azasteroid compound. For example, finasteride, is marketed by Merck & Co., Inc. under the registered trademarks Proscar and Propecia. A typical Proscar tablet contains 5 mg offinasteride as well as the following inactives: docusate sodium, FD&C Blue 2(aluminum lake), hydrous lactose, hydroxypropyl cellulose (LF grade), hydroxypropylmethyl cellulose, magnesium stearate, microcrystalline cellulose, pregelatinized starch, purified water, sodium starch glycolate, talc, titanium dioxide and yellow iron oxide. In contrast, the daily dosage of Prepecia is lower than Proscar and comprises, in addition to the finasteride, colloidal silicon dioxide, corn starch, docusate sodium benzoate, hydroxypropyl methylcellulose, iron oxide red (E172), lactose monohydrate, magnesium stearate, polyethylene glycol (PEG 6000), sodium starch glycolate and titanium dioxide. Proscar is also available in 1 mg tablet form which is used more for hair growth where the 5 mg is indicated for benign prostate hyperplasia (BPH).

In contrast, the inventors have established a topical finasteride foam for application to the user's scalp based upon a finasteride foam, for example a 0.1% finasteride foam, as defined by Table 1, which is generated from a stock finasteride solution, e.g. a 2% stock solution, as defined by Table 2.

TABLE 1
0.1% Finasteride Foam

| Ingredient | Quantity | Unit |
|---|---|---|
| Finasteride, 2% Stock Solution | 2.50 | mL |
| Medisca Foamil Base | 45.0 | mL |
| Medisca Foamil Base | q.s. to 50.0 | mL |

TABLE 2
2% Stock Solution

| Ingredient | Quantity | Unit |
|---|---|---|
| Finasteride, USP | 0.100 | g |
| Alcohol (99% USP) | 4.5 | mL |
| Alcohol (99% USP) | q.s. to 5.0 | mL |

Exemplary Finasteride Foam Preparation Process

Process 1: Finasteride 2% Stock Liquid Solution Preparation

An exemplary process for the preparation of a 2% stock liquid solution according to an embodiment of the invention comprises the following steps:
Step 1A: Triturate the finasteride (0.100 g) to form a fine, homogenous powder. Then sieve the powder 3 times to achieve the smallest particle size using a 40 to 50 mesh sieve
Step 1B: Incrementally add the fine, homogenous powder from step (1A) to the ISO propyl alcohol (99% USP) (4.5 Ml). The amount of alcohol used will be determined in dependence upon how much finasteride is employed.
Step 1C: Continuously mix until all solid particles have completely dissolved, and a homogenous liquid-like solution is obtained.
Step 1D: Add additional alcohol (99% USP) to the mixture from steps (1B) and (1C) to fill the required batch size (5.0 Ml)

Process 2: Powder to Medium Preparation

An exemplary process for the preparation of the medium from the powder according to an embodiment of the invention comprises the following steps:
Step 2A: Triturate the finasteride to form a fine, homogenous powder.
Step 2B: Incrementally add the fine, homogenous powder blend from step (2A) into the Foamil base (45.0 Ml plus processing error adjustments).
Step 2C: Continuously mix until all solid particles have completely dissolved and a homogenous liquid-like solution is obtained.
Step 2D: Spin (MAZ) at 2,000 rpm for 2 minutes

Process 3: Liquid to Medium Preparation

An exemplary process for the addition of the 2% finasteride solution to the medium according to an embodiment of the invention comprises the following steps:
Step 3A: Incrementally add the homogenous liquid-like solution from Process 2 into the Finasteride 2% Stock Solution from Process 1 (2.50 Ml plus processing error adjustments).
Step 3B. Continuously mix until all solid particles have completely dissolved and a homogenous liquid-like solution is obtained.

Process 4: Filling to Volume

An exemplary process for the preparation of final foam at the required volume according to an embodiment of the invention comprises the following steps:
Step 4A: Add additional Foamil Base to the homogenous liquid-like solution from Process 3 to fill to the required batch size (50.0 Ml plus processing error adjustments).
Step 4B: Continuously mix until all solid particles have completely dissolved and a homogenous liquid-like solution is obtained.

Process 5: Product Transfer

An exemplary process for the preparation of a 2% stock liquid solution according to an embodiment of the invention comprises the following steps:
Step 5A: Transfer final product from Process 4 to designated dispensing container(s).

Process 6: Product Labelling

Once the foam-based finasteride is packaged in the dispensing container(s) then the dispensing container(s) should be labelled in compliance with the product labelling, drug regulations, etc. in the jurisdiction that the topical finasteride foam will be sold. For example, the product labelling may include for example the following elements as per United States pharmacopeia (USP Chapter 795).

| | |
|---|---|
| Estimated | 30 days. |
| Beyond-Use Date | |
| Label 1 | Use as prescribed. Do not exceed prescribed dose. |
| Label 2 | Keep out of the reach of children. Not to be used |
| | by pregnant women or women breast-feeding. |
| Label 3 | Cap tightly after use. |
| Label 4 | For external use only. |
| Storage Label 1 | Room temperature storage (20° C.-23° C.) |
| Storage Label 2 | Protect from light |

Add auxiliary labels specific to the API to the dispensing container as deemed necessary including but not limited to:
Add auxiliary labels specific to the API to the dispensing container as deemed necessary including but not limited to:
Contact the pharmacist in the event of adverse reactions.
Consult health care practitioner if any prescription or over-the-counter medications are currently being used or are prescribed for future use.
Do not take with alcohol, sleep aids, tranquilizers, or other CNS depressants.
The quantity of the API administered is directly dependent on the quantity of the product applied.

Whilst the exemplary embodiment of the invention described supra exploits a foam as part of the pharmaceutical composition in combination with finasteride it would be evident to one of skill in the art that such foam-based compositions may comprise one or more 5?-reductase inhibitors together with one or more pharmaceutically acceptable excipients as required to prepare a dosage form for the effective delivery of the active agent. Accordingly, a topical finasteride composition may exploit a foam. Accordingly, alternate pharmaceutical compositions according to embodiments of the invention may exploit solutions, ointments, creams, gels, lotions, suspensions, and sprays as well as foams. Delivery of the 5?-reductase such as finasteride may exploit microspheres, microemulsions, nanoemulsions, nanoparticles, nanosuspensions, dermal sticks, roll-ons, pumps, patches, tapes, and the like.

Accordingly, a topical finasteride composition such as the foam means a composition that is applied onto the skin surface. Such a topical finasteride composition may act “locally” or “transdermally”. A transdermal composition refers to a composition that can be applied to the body surface, which then may permeate through the stratum corneum or scalp to form a reservoir just beneath the skin surface or may be absorbed systemically to provide desirable drug levels in the circulation. Such a reservoir being a “skin depot” or “depot” for the pharmaceutical composition that provides storage of drug within the skin and releases the contained drug to surrounding tissue over a prolonged period of time, and/or delayed to commence after a period of time.

Within embodiments of the invention the topical formulation may include a penetration enhancement or permeation enhancement as a means to increase the permeability of a biological membrane (i.e. scalp) to a drug, e.g. finasteride, so as to increase the rate at which the finasteride is transported through the membrane.

Within embodiments of the invention suitable carriers for the topical finasteride composition may include, but are not limited to, paraffin oils; esters of C8-C18 organic acids such as isopropyl myristate for example; C8-C30 fatty alcohols; silicone oils; vegetable oils; fractionated or hydrogenated vegetable oils; monoglycerides; diglycerides; triglycerides; phospholipids; dimethyl isosorbide; volatile solvents; N-methylpyrrolidone; N,N-dimethylacetamide and N,N-dimethylformamide; dimethylsulphoxide; alcohols such as ethanol and isopropyl alcohol; glycols such as propylene glycol, polyethylene glycol and glycerol; cyclodextrins such as beta-cyclodextrin, beta-hydroxy cyclodextrin, gamma-cyclodextrin, and hydroxypropyl cyclodextrin; and any mixture or mixtures thereof.

Within embodiments of the invention other pharmaceutically suitable excipients include, but are not limited to, surfactants, co-surfactants, penetration enhancers, antioxidants, buffering agents, preservatives, viscosity modifying agents, chelating/complexing agents, coloring agents, perfumes, polymers, gelling agents, alcohols, liquid or semi-solid oily components, and any mixture or mixtures thereof.

These other excipients according to embodiments of the invention can serve more than one purpose, such as, for example, a surfactant or co-surfactant used in the present invention can also act as a penetration or permeation enhancer.

Pharmaceutical compositions according to embodiments of the invention may comprise at least one 5α-reductase inhibitor, such as finasteride, as an active agent, one or more volatile solvents, one or more surfactants, optionally at least one penetration enhancer, and optionally one or more other pharmaceutically acceptable excipients.

According to embodiments of the invention the drug is dissolved or dispersed in a suitable pharmaceutically acceptable carrier. For example, when a composition of the present invention is in the form of a dispersion, it comprises at least one surfactant and optionally one or more co-surfactants along with a pharmaceutically acceptable carrier. A surfactant may be an anionic, cationic, non-ionic, or zwitterionic.

Suitable surfactants for use within a topical composition according to embodiments of the invention may include, but not be limited to, sodium laurate, sodium stearate, sodium lauryl sulfate, cetyl trimethyl ammonium bromide, benzalkonium chloride, a poloxamer (for example 231, 182, and 184 where the first digit (two digits in a three-digit number) in the numerical designation, multiplied by 300, indicates the approximate molecular weight of the hydrophobe; and the last digit×10 gives the percentage polyoxyethylene content), a polyoxyethylene sorbitan ester, lecithin, and any mixture or mixtures thereof.

Other suitable surfactants for use within a topical composition according to embodiments of the invention may include, but not be limited to, glycerol fatty acid esters such as glycerol monostearate, glycol fatty acid esters such as propylene glycol monostearate, polyhydric alcohol fatty acid esters such as polyethylene glycol monooleate, polyoxyethylene fatty acid esters such as polyoxyethylene stearate, polyoxyethylene fatty alcohol ethers such as polyoxyethylene stearyl ether, polyoxyethylene sorbitan fatty acid esters such as polyoxyethylene sorbitan monostearate, sorbitan esters such as sorbitan monostearate, alkyl glycosides such as cetearyl glucoside, sulfated oils such as a sulfuric ester of ricinoleic acid disodium salt, and sulfonated compounds such as alkyl sulfonates including sodium cetane sulfonate, amide sulfonates such as sodium N-methyl-N-oleyl laurate, sulfonated dibasic acid esters such as sodium dioctyl sulfosuccinate, alkyl aryl sulfonates such as sodium dodecylbenzene sulfonate, alkyl naphthalene sulfonates such a sodium isopropyl naphthalene sulfonate, a petroleum sulfonate such as aryl naphthalene with alkyl substitutes. Examples of suitable cationic surfactants include amine salts such as octadecyl ammonium chloride.

Suitable penetration enhancers for use within a topical composition according to embodiments of the invention may include, but not be limited to, sulfoxides such as dimethylsulfoxide (DMSO) and decylmethylsulfoxide (C10 MSO); ethers such as diethylene glycol monoethyl ether (available commercially as Transcutol™) and diethylene glycol monomethyl ether; 1-substituted azacycloheptan-2-ones, such as 1-n-dodecyl-cyclazacycloheptan-2-one; alcohols such as propanol, octanol, benzyl alcohol, and the like; fatty acids such as lauric acid, oleic acid, and valeric acid; fatty acid esters such as isopropyl myristate, isopropylpalmitate, methylpropionate, and ethyl oleate; polyol esters such as butanediol and polyethylene glycol monolaurate, amides and other nitrogenous compounds such as urea, N,N-dimethylacetamide (DMA), N,N-dimethylformamide (DMF), 2-pyrrolidone, 1-methyl-2-pyrrolidone, ethanolamine, diethanolamine, and triethanolamine; terpenes and terpinoids; alkanones; organic acids, such as salicylic acid and salicylates, citric acid and succinic-acid and the like; and any mixture or mixtures thereof.

Antioxidants for use within a topical composition according to embodiments of the invention may include, but not be limited to, for use within a topical composition according to embodiments of the invention may include, but not be limited to, tocopherol succinate, ascorbic acid, propyl gallate, vitamin E, butylated hydroxytoluene, butylated hydroxyanisole, sodium pyrosulfite, kojic acid, cysteine, hydroquinone, and the like, including any mixture or mixtures thereof.

Buffering agents for use within a topical composition according to embodiments of the invention may include, but not be limited to, alkali metal salts such as potassium and sodium carbonates, acetates, borates, phosphates, citrates and hydroxides; weak acids such as acetic, boric and phosphoric acids, and the like; and mixture or mixtures thereof.

Preservatives for use within a topical composition according to embodiments of the invention may include, but not be limited to, diazolidinyl urea and imidazolidinyl urea, as well as methyl, ethyl, propyl and butyl esters of p-hydroxybenzoic acid (parabens), isothiazolones, and the like, including any mixtures thereof.

Viscosity modifying agents for use within a topical composition according to embodiments of the invention may include, but not be limited to, cetyl alcohol, glycerol, polyethylene glycol (PEG), PEG-stearate, xanthan gums such as Keltrol™, and the like, including any mixture or mixtures thereof.

Chelating or complexing agents for use within a topical composition according to embodiments of the invention may include, but not be limited to, ethylenediaminetetraacetic acid (EDTA) and its derivatives, thioglycolic acid, thiolactic acid, thioglycerol, and the like, including mixture or mixtures thereof.

Polymers for use within a topical composition according to embodiments of the invention may include, but not be limited to, bioadhesive agents, gelling agents, film-forming agents, phase change agents, and any mixtures thereof. Examples of polymers include, but are not limited to, ethylcelluloses, acrylates, methacrylates, pyrrolidone polymers, including polymers of N-vinylpyrrolidone, polyoxyethylenes, hydroxypropyl methylcelluloses, hydroxypropyl celluloses, polymethylmethacrylates, cellulose acetates and their derivatives, cellulose acetate phthalates, hydroxypropyl methylcellulose phthalates, shellac, methacrylic acid based polymers such as those sold under the trademark EUDRAGIT™, zein, polycarbonates, polyorthoesters, polydioxanones, polyacetals, polyhydroxybutyrates, polyhydroxy valerates, polyethers, polyphosphazenes, polyhydroxy celluloses, polyalkylene oxalates, polyorthocarbonates, polyphosphoesters, star-branched polymers and copolymers, polysaccharides, polyketals, polyalkylene succinates, polypropylene oxides, chitin, chitosan, and other polymers known to a person skilled in the art of drug delivery, including copolymers, terpolymers, combinations and the likes, and any mixtures thereof.

Gelling agents for use within a topical composition according to embodiments of the invention may include, but not be limited to, cellulose and its derivatives, such as sodium carboxymethyl celluloses and hydroxyalkyl and alkyl celluloses, carbomers such as Carbopol™ and their derivatives, carob, carregeenans and derivatives, xanthan gum, sclerane gum, long chain alkanolamides, bentone and derivatives, kaolin, green clay, bentonite, magnesium aluminum silicate (Veegum™), guar gums (such as Jaguar™ HP-120), cross-linked acrylic acid polymers, and the like, including any mixtures thereof.

Liquid oily components for use within a topical composition according to embodiments of the invention may include, but not be limited to, sunflower oil, soybean oil, peanut oil, canola oil, cottonseed oil, coconut oil, palm oil, palm kernel oil, corn oil, flax seed oil, olive oil, safflower oil, fish oil, liquid state triglyceride esters of fatty acids, and the like, including any mixtures thereof.

Semi-solid or solid oily components for use within a topical composition according to embodiments of the invention may include, but not be limited to, C12-C30 higher fatty acids, e.g., stearic acid and linoleic acid, solid state mono-, di- and tri-glyceride esters of fatty acids, higher saturated alcohols, including aliphatic alcohols having 14-30 carbon atoms such as cetostearyl alcohol, waxes, such as carnauba wax, hydrocarbons, such as soft and hard paraffins, sphingolipids, and the like, including any mixtures thereof.

In embodiments of the present invention, pharmaceutical compositions provide topical delivery of 5α-reductase inhibitors such as finasteride to enhance the availability of the active agent to the hair follicles in the scalp, particularly when applied onto the scalp.

In embodiments, pharmaceutical compositions of the present invention, upon administration, permit the drug to penetrate through the skin or the scalp and thereby provide pharmacologically effective systemic drug levels.

In embodiments, compositions of the present invention form a depot or a reservoir at or near the point of application, and exhibit a pharmacological effect for an extended duration of time, and/or commencing after a delayed time, after application.

Further, compositions of the present invention are easy to formulate and frequently are removable by water washing. Also, in aspects, the compositions of the present invention have appreciable spreadability and can be easily applied to the skin.

Pharmaceutical compositions of the present invention comprising at least one 5α-reductase inhibitor as an active agent, can additionally comprise at least one another active agent. Such other active agents can either enhance or potentiate the activity of a 5α-reductase inhibitor or are useful for management (prophylaxis, amelioration or treatment) of any associated diseases/disorders, for which said 5α-reductase inhibitors are indicated. In certain embodiments, such additional active agents may be chemical compounds or extracts of one or more active components obtained from a natural source, such as plant extracts.

Such additional active agents include but are not limited to: hair loss preventing agents; hair growth promoting agents; anti-alopecia agents such as alfatradiol, dutasteride, epristeride, bexlosteride, izonsteride, lapisteride, turosteride, 17β-benzoyl-4-aza-5 alpha-androst-1-ene-3-one, 16-((4-chlorophenyl)oxy)-4,7-dimethyl-4-azaandronstan-3-one, 17β-Carboxy-4-androsten-3-one, steroidal oximes and saw palmetto extract, FCE 28260, and minoxidil; anti-infectives; antibacterials; antifungals; antihistaminics; immunomodulatory agents; anti-dandruff agents; antivirals; antiandrogenic agents such as fluconazole, ketoconazole and spironolactone; hormones; steroids; and the like.

Rapidly Dissolving Sidenafil Formats for Sublingual or Buccal Mucosa Delivery

The oral administration of drugs is by far the most common method of delivery. When administered orally, drug absorption usually occurs due to the transport of cells across the membranes of the epithelial cells within the gastrointestinal tract. Absorption after oral administration may be confounded by numerous factors. These factors include differences down the alimentary cannel in: the luminal pH; surface area per luminal volume; perfusion of tissue, bile, and mucus flow; and the epithelial membranes.

In contrast, oral transmucosal administration via the sublingual or buccal routes is generally quite rapid because of the rich vascular supply within the oral mucosa. The oral cavity exhibits a minimal barrier to drug transport and results in a rapid rise in blood concentrations. In addition, for significant drug absorption to occur across the oral mucosa, the drug must have a prolonged exposure to the mucosal surface. Taste, then, is one of the major determinants of contact time with the oral mucosa. Such is the case with most medicaments. Sildenafil citrate, which has proven to be a particularly effective drug for erectile disfunction and pulmonary arterial hypertension, exhibits a bitter taste.

A further issue affecting the absorption of orally administered drugs is the form of the drug. Most orally administered drugs are in the form of tablets or capsules. This is done primarily for convenience, economy, stability, and patient acceptance. Accordingly, these capsules or tablets must be disintegrated or dissolved before absorption can occur. There are a variety of factors capable of varying or retarding disintegration of solid dosage forms. Further, there are a variety of factors that affect the dissolution rate and therefore determine the availability of the drug for absorption.

Although the tableted dosage form of sildenafil citrate has been proven very effective for male erectile dysfunction, such a dosage form has shown certain side effects. For instance, some patients taking sildenafil citrate in the tablet formed marketed under the trademark Viagra experienced gastroesophageal reflux, a regurgitation of food from the stomach. Accordingly, a gradual release dosage form of sildenafil citrate might prove to be gentler to the stomach and enhance the desirability of the drug to a larger patient population requiring its use but increases the complexity and cost due to the requirement for a capsule or tablet coating that either degrades slowly or supports slow release of the drug.

Not only is drug absorption a central issue in drug delivery, but also the bioavailability of the drug as well. Bioavailability is defined as the rate at which and the extent to which the active moiety (drug or metabolite) enters the general circulation, thereby gaining access to the site of action. Bioavailability depends upon a number of factors, including how a drug product is designed and manufactured, its physiochemical properties, and factors that relate to the physiology and pathology of the patient.

When a drug rapidly dissolves from a dosage form and readily passes across membranes, absorption from most administration sites tends to be complete. This is not always the case for drugs given orally. Before reaching the vena cava, the drug must move down the alimentary cannel and pass through the gut wall and liver, which are common sites of drug metabolism. Thus, the drug may be metabolized before it can be measured in the general circulation. This can cause a decrease in the amount of drug available to exert a therapeutic effect called the first pass effect. A large number of drugs show low bioavailabilities owing to an extensive first pass metabolism. The two other most frequent causes of low bioavailability are insufficient time in the oral cavity, or gastrointestinal tract and the presence of competing reactions. Differences in bioavailability can have profound clinical significance. Sildenafil citrate, for example, exhibits only a 40% bioavailability after oral administration, of which the active metabolite accounts for about one-half.

For the typical tablet, its formulation and manufacturing process are a tradeoff between two almost mutually exclusive properties: the ability to rapidly dissolve and hardness. Usually, when the ease of dissolution (i.e., the rapidity of disintegration) of the tablet is increased, the hardness of the tablet decreases. The invention provides a new formulation and manufacturing process that overcomes these previous limitations and that achieves the optimal balance between physical properties needed for manufacturing and handling, e.g., hardness, and rapid disintegration (dissolving) characteristics in vivo, in addition to other advantageous properties, such as being free of organic solvents and having an improved, smooth tactile effect (“mouth feel”).

Beneficially, embodiments of the invention advantageously provide for sublingual and buccal mucosa delivery formats that are uniform in formulation, economical to produce, and provide for a delivery format allowing sildenafil citrate, for example, to pass rapidly into the patient's bloodstream through the sublingual or buccal routes. For example, such delivery formats may include a rapidly dissolving tablet for sublingual, a rapidly dissolving lollipop for buccal mucosa delivery, and a rapidly dissolving chewable tablet or soft candy.

The formulation and method of manufacture of these compressed tablets, lollipops or candies results in a tablet, lollipop or candy that is hard, and resistant to breakage during handling allowing their packaging, shipping and handling with low breakage rates. Though the tablets and lollipops are advantageously hard, they also rapidly disintegrate when they are contacted by body fluid or other aqueous medium. The tablets and lollipops described here also have a greatly improved taste relative to the drug's normal taste making the tablets significantly more palatable to the consumer.

The invention provides a physiologically acceptable tablet, lollipop or candy comprising a compressed tablet, lollipop or candy formulation free of organic solvent residue that rapidly disintegrates when placed in a body cavity, that comprises at least one water soluble non-saccharide polymer. Within embodiments of the invention the non-saccharide, water soluble polymer may include, but not be limited to, polyvinylpyrrolidone (PVP), polyethylene glycol, sodium alginate, hydroxypropyl cellulose, hydroxypropylmethyl cellulose, hydroxyethyl cellulose and a mix or mixes thereof. The non-saccharide, water soluble polymer can be between about 0.5% to about 20% of the dry weight of the tablet, lollipop or candy.

Within embodiments of the invention the PVP may include, but not be limited to, N-vinyl pyrrolidone, 3-methyl N-vinylpyrrolidone, N-vinyl amide pyrrolidone, N-vinyl acetate pyrrolidone, vinylpyrrolidone-vinyl acetate copolymer, and acrylamide-vinylpyrrolidone co-polymer and a mix or mixes thereof.

A tablet, lollipop or candy according to embodiments of the invention may also comprise a formulation that further comprises a saccharide of low moldability. The low moldable saccharide may be mannitol, lactose, glucose, sucrose, lactitol, or a mixture thereof. The saccharide of low moldability may vary within embodiments from a low percentage of the weight of the tablet, lollipop or candy to a high percentage of the weight of the tablet, lollipop or candy.

A tablet, lollipop or candy according to an embodiment of the invention may also comprise a saccharide of high moldability. The saccharide of high moldability can be maltose, maltitol, sorbitol, or a mixture thereof. The saccharide of high moldability may similarly vary within embodiments from a low percentage of the weight of the tablet, lollipop or candy to a high percentage of the weight of the tablet, lollipop or candy.

The invention also provides a tablet further comprising a pharmaceutically active ingredient within a matrix. The formulation of the invention can further comprise at least one additive agent selected from the group consisting of a disintegrant, a flavourant, an artificial sweetener, a perfume, and a colorant. The matrix may provide for a rapidly dissolving tablet.

The invention also provides a lollipop further comprising a pharmaceutically active ingredient within a matrix molded about a handle (e.g. stick) which is operable to position the medicine adjacent a selected area of the patient's mucosa. The formulation of the invention can further comprise at least one additive agent selected from the group consisting of a disintegrant, a flavourant, an artificial sweetener, a perfume, and a colorant. The matrix may provide for a rapidly dissolving lollipop such that the patient licking or sucking the lollipop for a predetermined time period dissolves the lollipop and delivers the pharmaceutically active ingredient to the patent's buccal mucosa.

The invention also provides a tablet further comprising a pharmaceutically active ingredient within a candy matrix. The formulation of the invention can further comprise at least one additive agent selected from the group consisting of a disintegrant, a flavourant, an artificial sweetener, a perfume, and a colorant. The matrix may provide for a rapidly dissolving candy.

The invention also provides a chewable candy further comprising a pharmaceutically active ingredient within a matrix. The formulation of the invention can further comprise at least one additive agent selected from the group consisting of a disintegrant, a flavourant, an artificial sweetener, a perfume, and a colorant. The matrix may be a soft candy to provide for a physiologically acceptable chewable or suckable soft candy.

Within embodiments of the invention the quantity of the pharmaceutical in the tablet, lollipop, soft candy etc. may be predetermined or the dose may be formulated based upon one or more basis such as the patient's weight and/or the patient's age.

Water Soluble Polymers as Binders in Sublingual or Buccal Mucosa Delivery Formats

A tablet, lollipop or candy formulation according to an embodiment of the invention may comprise a binder having at least one water-soluble or water dispersible polymer that is not a saccharide. In alternative embodiments, the non-saccharide, water soluble polymer can be polyvinylpyrrolidone, polyethylene glycol, gelatin, agar, sodium alginate (alginic acid and derivatives), hydroxypropyl cellulose, hydroxypropylmethyl cellulose, hydroxyethyl cellulose, cellulose derivatives in general, carboxyvinyl polymers, glucans, mannans, xantan gums, and the like. The tablet, lollipop or candy can contain any combination of these water-soluble, polymer binders.

The binders used in these tablets, lollipops or candies according to embodiments of the invention can also contain a mixture of water soluble, non-saccharide polymer(s) with another binder, such as a saccharide of high moldability, e.g., maltose, maltitol, sorbitol, or a mixture thereof or saccharides of low moldability can be included in the tablet, lollipop or candy formulation as fillers, as discussed supra.

In some embodiments, advantageous binder combinations can include a non-saccharide water soluble polymer and a saccharide having high moldability at a weight ratio of about 1:9 to about 9:1. The tablets, lollipops or candies of the invention are not limited by these ratios, and in some embodiments, they contain no saccharides of high moldability (saccharides of low moldability can also be optionally included in the tablet, lollipop or candy formulation).
Binders impart cohesiveness to the tablet, lollipop or candy and ensure tablet, lollipop or candy strength after compression. Water soluble binders are also important in the humidification step, discussed infra. The binder swells upon absorption of the water, allowing more thorough hydration of the other compositions of the formulation and deeper penetration of the water into the tablet, lollipop or candy. The non-saccharide, water soluble polymers also act as a disintegrant, contributing to the rapid disintegration properties of the tablets, lollipops or candies of the invention. The non-saccharide, water soluble polymers may also contribute to and enhance a “smooth feeling” of the tablet, lollipop or candy when it dissolves in the mouth.
Polyvinylpyrrolidone (“PVP”) may be a non-saccharide, water-soluble, polymer binder according to embodiments of the invention. In one embodiment, partial or complete replacement of the formulation's non-PVP water soluble polymer binder(s) by PVP allows a lower level of water insoluble lubricant, such as magnesium stearate, to be used in the formulation while retaining the properties of a “when the tablet, lollipop or candy dissolves in the mouth. Use of PVP also provides for a quicker in vivo disintegration/dissolving time. Use of PVP as the only binder in the formulation results in tablets, lollipops or candies with a better resistance to moisture (i.e., the tablets, lollipops or candies absorb less moisture after manufacture). Use of PVP in lieu of saccharides in the formulation has the same advantage as when using other synthetic polymers versus materials from a natural source, i.e. lower contaminant levels and prices. PVP can be any polymer of vinylpyrrolidone.

In various embodiments of the invention, the amount of PVP in the formulation may be between about 0.5% to about 10% of the volume of the tablet, lollipop or candy but other percentages are possible. In one embodiment PVP is about 5% of the volume. In one embodiment, PVP is used as the sole binder in a formulation, at a total of about 5% of the formulation. In alternative embodiments, PVP is used in varying amounts with at least one other binder, such as, e.g., a water-soluble polymer such as polyethylene glycol, sodium alginate, hydroxypropyl cellulose, hydroxypropylmethyl cellulose, hydroxyethyl cellulose, or a saccharide of high moldability, e.g., maltose. Exemplary binder combinations may include, but not be limited to, about 0.5% of PVP and about 4.5% maltose; about 1.5% of PVP and about 3.5% maltose; about 2.5% of PVP and about 2.5% maltose; about 3.0% of PVP and about 2.0% maltose, or any other combination of maltose and PVP, or equivalent compositions, thereof.

Saccharides of Low Moldability as Fillers in Sublingual or Buccal Mucosa Delivery Formulations

The invention optionally provides for a tablet, lollipop or candy formulation comprising a single or a mixture of two saccharides of low moldability, such as mannitol, erythritol, xylitol, lactose, glucose, sucrose, lactitol, or other similar saccharides or derivatives thereof of low moldability, or a mixture thereof, as filler in the tablet, lollipop or candy formulation. In different embodiments, the saccharide of low moldability is about 40% to about 99% of the weight of the tablet, lollipop or candy although other percentages are possible.

No Organic Solvents Used in Sublingual or Buccal Mucosa Delivery Formulations

Tablet, lollipop or candy manufacturing processes frequently use organic solvents, leaving an organic solvent residue in the final tablet, lollipop or candy product. Accordingly, methods within embodiments of the invention may not utilize an organic solvent in the manufacturing process. This results in a finished tablet, lollipop or candy completely lacking in any organic solvent residue.

Soft Buccal Mucosa Delivery Formulations

A soft candy according to embodiments of the invention may comprise in addition to the pharmaceutically active ingredient or combination of pharmaceutically active ingredient to be absorbed through the buccal mucosa including, but not limited to;
a water-soluble protein having an absorption-promoting action;
a polyhydric alcohol; and
a fatty acid ester or/and a carboxyvinyl polymer.

A water-soluble protein to be incorporated in the soft candy according to the present invention, there may be used any water-soluble protein capable of promoting the absorption of drugs. Such water-soluble protein includes natural proteins of animal or plant origin and non-natural ones which are artificially-produced peptides. The term “protein” as used herein thus includes peptides in view of their effects in practicing the present invention, although peptides are distinguished from proteins in some technical fields. Natural proteins usable in the practice of the present invention are gelatin, solubilized collagen, casein, glue, and hydrolysates therefrom. Among them, preferred are animal proteins such as gelatin and solubilized collagen. Usable gelatin species have a molecular weight of tens of thousands to hundreds of thousands and include acid process gelatin species and alkali process gelatin species respectively produced by acid and alkali hydrolysis of proteins contained in the bone, skin, tendon and the like of animals. Usable collagen species are solubilization products from insoluble collagen by partial hydrolysis and chemically modified collagen species such as maleic, succinic or phthalic anhydride addition products. Their molecular weight lies in almost the same range as that for the gelatin species. Furthermore, as the above-mentioned peptides usable in the practice of the present invention, there may be mentioned peptides produced by combining amino acids of the same or different kinds by a synthetic or semisynthetic technique. Examples of such peptides are polyalanine, polylysine, polyglutamine and other amino acid homopolymers, and copolymers of different amino acids as produced by combining various amino acids (neutral, basic, acidic) in a desired sequence. In most cases, they have a smaller molecular weight as compared with the above-mentioned natural proteins, for example several hundred to several thousand. In practicing the invention, the above-mentioned water-soluble proteins are used either singly or as a mixture of two or more of them.

A pharmaceutically acceptable polyhydric alcohol is used as the third component for the purpose of controlling and maintaining the softness of the soft buccal and, also, of controlling the rate of dissolution or disintegration. The above-mentioned polyhydric alcohol includes, typically and among others, glycols, triols and polyols, for example glycols of 2 to 6 carbon atoms (e.g. ethylene glycol, propylene glycol, butylene glycol), polyethylene glycol (with an average molecular weight of about 200 to 50,000, and preferably of about 300 to 5,000), etc., and triols such as glycerin, trimethylolpropane, etc., and polyols such as polyvinyl alcohol, etc. In addition, cellulose, sugars, etc. also fall within the scope of the polyhydric alcohol that can be employed in the practice of this invention. The cellulose may be one having an average molecular weight of about 13,000 to 400,000 and preferably of about 40,000 to 200,000. Thus, for example, alkyl- and hydroxyalkylcelluloses whose alkyl moieties contain 1 to 4 carbon atoms (e.g. methylcellulose, ethylcellulose, propylcellulose, hydroxymethylcellulose, hydroxyethylcellulose, etc.), and carboxymethyl cellulose and alkali metal (e.g. sodium, potassium, etc.) salts thereof. Typical of said sugars are monosaccharides, disaccharides and polysaccharides. The monosaccharides preferably contain 2 to 6 carbon atoms and include, for example, glucose, galactose, fructose, mannose, mannitol, sorbitol, etc., and the disaccharides may be the dimers of such monosaccharides, for example maltose, lactose, sucrose, etc. The polysaccharides include the genuine polysaccharides which are condensates of at least 7 units of the above-mentioned monosacharides, such as starch and its derivatives (e.g. carboxymethyl-starch, hydroxypropyl-starch), dextrin, dextran, chitin, alginic acid, glycogen, and composite condensates of at least one of the above-mentioned monosaccharides with one or more non-sugar substances such as mannan, pectin, gum arabic, etc. The above-mentioned third component are used either singly or as a mixture of two or more of them. A polyhydric alcohol is preferably a non-volatile compound to give plasticity such as glycerin are advantageous in many instances.

The above-mentioned fatty acid ester is a monoalcohol or polyalcohol ester of a saturated or unsaturated fatty acid. More specifically, such ester consists of a saturated or unsaturated fatty acid containing 4 to 30 carbon atoms, preferably 8 to 20 carbon atoms, as the carboxylic acid moiety (the number of double bonds in the unsaturated fatty acid is about 1 to 8) and a monoalcohol or polyalcohol containing 1 to 30 carbon atoms, preferably 1 to 20 carbon atoms, as the ester residue moiety. This polyalcohol may be the same as the above-mentioned polyhydric alcohol but preferably contains about 2 to 10 hydroxy groups (e.g. glycerin, ethylene glycol, propylene glycol, etc.). Examples of the above-mentioned fatty acid ester include alkyl esters of intermediate or higher fatty acids containing 8 to 20 carbon atoms (e.g. isopropyl myristate), and glycerin esters of fatty acids containing 1 to 20 carbon atoms (inclusive of mono-, di- and triglycerides), sucrose fatty acid esters whose fatty acid moieties contain 9 to 30 carbon atoms (e.g. mixtures of mono-, di- and triesters of stearic acid and palmitic acid), etc. The fatty acid esters include not only such monocarboxylic acid esters as mentioned above but also those consisting of a dicarboxylic acid of 4 to 20 carbon atoms and the ester moiety described above (e.g. diisopropyl adipate, diethyl sebacate, etc.). The carboxyvinyl polymer mentioned may have an average molecular weight of about 40,000 to 3,000,000, and includes polyacrylic acid and polymethacrylic acid and their salts, for instance. Within embodiments of the invention it is desirable to choose a fatty acid ester which is capable of dispersing or dissolving the medicament. The above-mentioned components are used either singly or as a mixture of two or more of them.

In addition to the above components, there may also be incorporated other additives selected from among the various pharmaceutically acceptable additives available to those skilled in the art for the purpose of assisting in the development of characteristics of the soft buccal, of improving the processability, moldability and quality of the preparation, of enhancing the dispersability and stability of the medicament, for instance. Such additives are other than those mentioned as the essential components and include the following substances.
Flavors (saccharin sodium, glycyrrhizin, malt syrup, citric acid, tartaric acid, menthol, lemon oil, citrus flavor, common salt, etc.);
Stabilizers/preservatives (parahydroxybenzoic acid alkyl esters, antioxidants, antifungal agents, etc.);
Colors (water-soluble tar colors, natural colors, titanium oxide, etc.);
Excipients/disintegration adjusting agents (magnesium silicate, light silicic acid anhydride, synthetic aluminum silicate, precipitated calcium carbonate, magnesium aluminum metacilicate, calcium hydrogen phosphate, etc.);
Water-soluble polymers other than water-soluble proteins (natural polymers, synthetic polymers, etc.); and
Stearic acid and its salts, talc, palmitic acid, and other substances known as emulsifiers, dispersants, binders, thickeners, etc.

Other Formulation Components

In some embodiment of the invention, the tablets, lollipops or candy may optionally include an insoluble lubricant, such as magnesium stearate, or a derivative thereof. In various embodiments, the magnesium stearate is between 0.1% and 2.0%, or between 0.5% and 1.0%, of the weight of the tablet, lollipop or candy. Lubricants help in the manufacturing of the tablet, lollipop or candy; e.g., they help prevent “ejection sticking” of compressed formulation to the pressing dies and punches, for example.

In another embodiment, a disintegrant other than the non-saccharide water soluble polymers described herein, such as PVP, is added to the formulation to further enhance the rate of disintegration. Disintegrants within embodiments of the invention may include but not be limited to croscarmellose sodium and sodium starch glycolate.

The tablets, lollipops or candies of the invention can further comprise another medicament, drug, palliative, nutritive, or pharmaceutically active material, e.g., a drug, medicament, nutrient, etc., can be added to the formulation of the invention in addition to a drug such as sildenafil citrate that acts to protect cyclic guanosine monophosphate (cGMP) from degradation by cGMP-specific phosphodiesterase type 5 (PDE5) in the corpus cavernosum. Such additional medicament, drug, palliative, nutritive, or pharmaceutically active materials may include, but not be limited to, antacids, gastrointestinal agents, analgesics, anti-infectives, central nervous system active agents, cardiovascular agents, cough therapies, vitamins, and other pharmaceutical, nutritional and dietary agents. In embodiments of the invention the particle size of the medicament, drug, palliative, nutritive, or pharmaceutically active material is in such a range that the “mouth feel,” or smooth tactile effect, of the final product (the tablet, lollipop or candy of the invention) in the oral cavity will not be negatively affected. Similarly, the taste of the medicament, drug, palliative, nutritive, or pharmaceutically active material does not override that of any flavouring or taste modifier added to the tablet, lollipop or candy or the flavouring added is modified to minimize the unpleasant taste of all medicament, drug, palliative, nutritive, or pharmaceutically active materials present within the tablets, lollipops or candies.

In particular, for medicament, drug, palliative, nutritive, or pharmaceutically active materials that have a bitter taste or stinging effect when given into the mouth, an extra taste-masking ingredient is generally added to ensure the good clinical acceptance of the tablets, lollipops or candies. Accordingly, colors, flavours and sweeteners can be included in the formulation to improve the overall organoleptic characteristics of the final product.

Any colorant can be used, as long as it is approved and certified by the appropriate regulatory authority or authorities, for example the U.S. Federal Drug Administration. Accordingly, colors within embodiments of the invention may include, but not be limited to, allura red, acid fuschin D, naphtalone red B, food orange 8, eosin Y, phyloxine B, erythrosine, natural red 4, carmine, to name a few. Adding color to a tablet, lollipop or candy formulation may be achieved by dissolving the dye in the binding solution prior to a granulating process, for example.

Within embodiments of the invention sweetening agents may added to the tablet, lollipop or candy formulation to create or add to the sweetness (which is afforded by the presence of saccharide fillers and binders, e.g., mannitol, lactose, and the like). For example, cyclamates, saccharin, aspartame, and acesulfame K may be used. Sweeteners other than sugars have the advantage of reducing the bulk volume of the tablet, lollipop or candy and not effecting the physical properties of the granulation. Within embodiments of the invention flavoured or unflavoured tablets, lollipops or candies may be sweetened with a natural sweetener or sweeteners and/or artificial sweetener or sweeteners. Artificial sweeteners, while contributing none or a minimal number of calories, can impart an aftertaste to a tablet, lollipop or candy flavored and sweetened by their inclusion. Additionally, different sweeteners impart their peak sweetness at different times and rates over the time such that a tablet, lollipop or candy which has dissolved stays in a consumer's mouth. The artificial sweeteners may be used in combination to level the sweetness sensation over time, and to minimize any potential aftertaste.

Use of natural sweeteners in combination with one or more artificial sweeteners can also be used to mitigate the aftertaste and peak sweetness issues. Natural or artificial sweeteners may also be used singly, rather than in combination. Suitable sweeteners that can be included are those well known in the art, including both natural and artificial sweeteners. Suitable sweeteners include, by example water-soluble sweetening agents, water-soluble artificial sweeteners, dipeptide based sweeteners, naturally occurring water soluble sweeteners, and protein based sweeteners.

Water-soluble sweetening agents may include, but not be limited to, monosaccharides, disaccharides and polysaccharides such as xylose, ribose, glucose (dextrose), mannose, galactose, fructose (levulose), sucrose (sugar), honey, maltose, invert sugar (a mixture of fructose and glucose derived from sucrose), partially hydrolyzed starch, corn syrup solids, dihydrochalcones, monellin, steviosides, and glycyrrhizin;

Water-soluble artificial sweeteners may include, but not be limited to, soluble saccharin salts, i.e., sodium or calcium saccharin salts, cyclamate salts, the sodium, ammonium or calcium salt of 3,4-dihydro-6-methyl-1,2,3- - -oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like;

Dipeptide based sweeteners may include, but not be limited to, L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5-dihydrophenylglycine, L-aspartyl-2,5-dihydro-L-phenylal-anine, L-aspartyl-L-(1-cyclohexyen)-alanine, and the like;

Water-soluble sweeteners may include, but not be limited to, those derived from naturally occurring water-soluble sweeteners, such as a chlorinated derivative of ordinary sugar (sucrose), known, for example, under the product description of sucralose; and

Protein based sweeteners may include, but not be limited to, thaumatoccous danielli (Thaumatin I and II).

Other sweeteners may be used as well.

Within embodiments of the invention a flavour component may comprise one or more natural or artificial flavours, or combinations thereof. By providing such flavours, and thereby flavour satisfaction, the tablet, lollipop or candy can encourage user compliance with taking the tablets, lollipops or candies where prescribed or increase overall user acceptance in non-prescribed use. Within embodiments of the invention the use of more than one flavour in a single tablet, lollipop or candy may be provided where such a multiplicity of flavour combinations enhances the user experience. Optionally, the tablet, lollipop or candy may be layered with different flavours with each layer bearing a color associated with its flavour. Alternatively, multiple flavours may be included in a tablet, lollipop or candy with no color differentiation apparent on the tablet, lollipop or candy. Optionally, tablets, lollipops or candies may be formed with discrete flavourings and marketed as a single flavor or marketed with multiple flavours per packaging container.

Suitable flavourings may include natural and/or artificial flavours. These flavourings may be chosen from synthetic flavour oils and flavouring aromatics, and/or oils, oleo resins and extracts derived from plants, leaves, flowers, fruits, vegetables, and so forth, and combinations thereof. Representative flavour oils may include, but not be limited to, spearmint oil, cinnamon oil, peppermint oil, clove oil, bay oil, thyme oil, cedar leaf oil, oil of nutmeg, oil of sage, and oil of bitter almonds. A flavouring may include, but not be limited to, artificial, natural or synthetic fruit flavours such as mango, vanilla, chocolate, coffee, cocoa and citrus oil, including lemon, orange, grape, lime and grapefruit and fruit essences including apple, pear, peach, strawberry, raspberry, cherry, plum, pineapple, apricot, rose hips, and so forth, and vegetable flavours such as tomato, cucumber, wheat grass, capsacinoids, and so forth. These flavourings can be used individually or in combination. may include, but not be limited to, mints such as peppermint, wintergreen, spearmint, birch, anise and such fruit flavours, as cherry, lemon, lime, orange, grape, artificial vanilla, and such spice and herb flavours such as cinnamon derivatives, ginger, lemongrass, basil, lavender, whether employed individually or in a mixture.

Flavourings such as aldehydes and esters including cinnamyl acetate, cinnamaldehyde, citral, diethylacetal, dihydrocarvyl acetate, eugenyl formate, p-methylanisole, and so forth may also be used. Generally, any flavouring or food additive, such as those described in Chemicals Used in Food Processing, publication 1274 by the National Academy of Sciences, pages 63-258, which is incorporated herein by reference, may be used. Further examples of aldehyde flavourings include, but are not limited to acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavours); butyraldehyde; citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof; and the like.

The amount of flavouring employed is normally a matter of preference subject to such factors as flavour type, individual flavour, and strength desired. Thus, the amount may be varied in order to obtain the result desired in the final product. Such variations are within the capabilities of those skilled in the art without the need for undue experimentation.

Within other embodiments of the invention biodegradeable microspheres or capsules or other biodegradeable polymer configurations capable of sustained delivery of a composition (e.g., a pharmaceutical) can be included in the tablets, lollipops or candies of the invention. This allows for the advantageous combination of a rapidly dissolving tablet, lollipop or candy (of the invention) releasing a second agent (the sustained delivery construction).

Exemplary Sildenafil Citrate Rapidly Dissolving Tablet Preparation Process

Within the exemplary process described below 3.36 g of sildenafil citrate is employed for the selective inhibitor of cGMP-specific phosphodiesterase type 5 (PDE5). Alternatively, this may be replaced with 2.40 g of sildenafil.

TABLE 3
Solid Suspension for 96 × 750 mg (0.93 mL) Tablets

| Ingredient | Quantity | Unit |
|---|---|---|
| Sildenafil Citrate USP | 3.360 | G |
| Mango Flavour (Powder) | 0.480 | G |
| Flavour (Powder) | 0.288 | G |
| Vanilla Extract (Powder) | 0.288 | G |
| Medi-RDT Base | TBD | |

Process 1: Mold Calibration

An exemplary process for the calibration of tablet molds according to an embodiment of the invention comprises the following steps:
Step 1A: Determine the required quantity of Medi-RDT Base for 96 tablets based on the actual size of tablet mold being used.

Process 2: Powder Preparation

An exemplary process for the powder preparation according to an embodiment of the invention comprises the following steps:
Step 2A: Pass the Medi-RDT Base through a 40 or 50 mesh sieve and weigh the required quantity (amount calculated in step 5A(iii))
Step 2B: By geometric addition, combine and triturate the following ingredients together to form a homogenous powder blend:

2B (1) Sildenafil citrate
2B (2) Mango Flavour (Powder)
2B (3) Raspberry Flavour (Powder)
2B (4) Vanilla Extract (Powder)

Step 2C: By geometric addition, combine and mix the following ingredients together to form a homogenous powder blend:

2C (1) Sieved Medi-RDT Base (from Step 2A)
2C (2) Homogenous powder blend (from Step 2B)
2C (3) Spin (MAZ) at 1,000 rpm for 30 seconds
Excessive force should be avoided as the Medi-RDT
Base should not be triturated.

Step 2D: Prior to filling the tablet mold cavities pass the homogenous powder blend from Step 2C through a 40 or 50 mesh sieve to improve flow properties and obtain content uniformity.

Process 3: Mold Filling and Heating

An exemplary process for the mold filling and heating according to an embodiment of the invention comprises the following steps:
Step 3A: Fill the 96 mold cavities by tapping and pressing the sieved homogenous powder blend from step 2D into the cavities using the upper part of the mold. Repeat at least three times to ensure the cavities are completely filled, adding additional powder blend as necessary.
Step 3B: Gently heat the powder blend by placing the filled base cavity plate of the mold to 225° C. for 10-15 minutes, avoiding overheating, using an appropriate oven to form a homogenous solid dispersion.

Process 4: Cooling

An exemplary process for the cooling of the molded tablets according to an embodiment of the invention comprises the following steps:
Step 4A: Carefully remove the tablet mold from the heated oven.
Step 4B: Leave to cool for approximately 1 minute and 45 seconds at room temperature.
Step 4C: Immediately remove the tablets by flipping over the mold onto a piece of wax or ointment paper and gently tapping the mold whilst holding it to avoid breaking shaking and breaking the tablets.
Step 4D: Allow tablets to cool for 30 minutes at controlled temperature and relative humidity.

Process 5: Validation Technique

An exemplary process for validation according to an embodiment of the invention comprises the following steps:
Step 5A: Weight 20 tablets separately once cool.
Step 5B: The final weight of each tablet from step (5A) should be between 90% and 110% of the theoretically calculated weight in accordance with USP guidelines.

Process 6: Product Transfer

An exemplary process for the transfer of the tablets according to an embodiment of the invention comprises the following steps:
Step 6A: Transfer the final product into the specified dispensing container.

Process 7: Product Labelling

Once the sildenafil citrate based rapid dissolving tablet is packaged in the dispensing container(s) then the dispensing container(s) should be labelled in compliance with the product labelling, drug regulations, etc. in the jurisdiction that the topical finasteride foam will be sold. For example, the product labelling may include for example the following elements:

| | |
|---|---|
| Estimated | 6 months as per USP. |
| Beyond-Use Date | |
| Label 1 | Use as prescribed. Do not exceed prescribed dose. |
| Label 2 | Keep out of the reach of children. |
| Label 3 | Cap tightly after use. |
| Label 4 | For external use only. |
| Label 5 | May impair mental and/or physical activity. Use |
| | care when operating a car or machinery. |
| Storage Label 1 | Room temperature storage (20° C.-23° C.). |
| Storage Label 2 | Protect from light and keep in a dry place. |

Add auxiliary labels specific to the API to the dispensing container as deemed necessary including but not limited to:
Contact the pharmacist in the event of adverse reactions.
Discard container after use.
Consult health care practitioner if any prescription or over-the-counter medications are currently being used or are prescribed for future use.
Do not take with alcohol, sleep aids, tranquilizers, or other CNS depressants.
The quantity of the API administered is directly dependent on the number of tablets taken.

It should be noted that the weight of the rapid dissolving tablet is mainly affected by factors such as compression force, flow properties of the powder mixture (which will therefore vary with the active pharmaceutical ingredient(s) and excipients in the formulation) and particle size distribution. The mixture must be sieved prior to filling to ensure appropriate maximum particle size. For a target RDT of 750 mg the acceptable range of measured tablet weight is 750 mg±30 mg or in the range 720 mg to 780 mg.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A pharmaceutical composition comprising:
a first component; and
a second component.

2. The pharmaceutical composition according to claim 1, wherein
the first component is a 5α-reductase inhibitor, and
the second component is a topical carrier for the 5α-reductase inhibitor.

3. The pharmaceutical composition according to claim 2, wherein
the 5α-reductase inhibitor is (5α,17β)-N-(1,1-dimethylethyl)-3-oxo-4-azaandrost-1-ene-17-carboxamide (finasteride).

4. The pharmaceutical composition according to claim 2, wherein
the 5α-reductase inhibitor is an antiandrogen.

5. The pharmaceutical composition according to claim 2, wherein
the 5α-reductase inhibitor is at least one of alfatradiol, dutasteride, epristeride, bexlosteride, izonsteride, lapisteride, turosteride, 17β-benzoyl-4-aza-5 alpha-androst-1-ene-3-one, 16-((4-chlorophenyl)oxy)-4,7-dimethyl-4-azaandronstan-3-one, 17β-Carboxy-4-androsten-3-one, steroidal oximes and saw palmetto extract.

6. The pharmaceutical composition according to claim 2, wherein
the topical carrier is a foam.

7. The pharmaceutical composition according to claim 1, wherein
the first component is an antihypertensive potassium channel opener, and
the second component is a topical carrier for the antihypertensive potassium channel opener.

8. The pharmaceutical composition according to claim 7, wherein
the antihypertensive potassium channel opener is minoxidil.

9. The pharmaceutical composition according to claim 7, wherein
the topical carrier is a foam.

10. The pharmaceutical composition according to claim 1, wherein
the first component is an inhibitor of cyclic guanosine monophosphate (cGMP) specific phosphodiesterase type 5 (PDE5); and
the second component is a non-pharmaceutical carrier allowing the cGMP inhibitor to be in tablet form for placement by a user into their mouth and providing for rapid dissolving allowing absorption of the cGMP inhibitor by the user to be primarily through their mouth.

11. The pharmaceutical composition according to claim 10, wherein
the absorption is at least one of sublingual and buccal.

12. The pharmaceutical composition according to claim 10, further comprising one or more flavourings.

13. The pharmaceutical composition according to claim 10, wherein
the non-pharmaceutical carrier is sugar free.

14. The pharmaceutical composition according to claim 10, wherein
the cGMP inhibitor is sildenafil citrate.

15. The pharmaceutical composition according to claim 10, wherein
the cGMP inhibitor is tadalafil or vardenafil.

16. The pharmaceutical composition according to claim 1, wherein
the first component is an inhibitor of cyclic guanosine monophosphate (cGMP) specific phosphodiesterase type 5 (PDE5); and
the second component is a non-pharmaceutical carrier allowing the cGMP inhibitor to be in a form for placement by a user into their mouth and providing for absorption of the cGMP inhibitor by the user to be primarily through their mouth.

17. The pharmaceutical composition according to claim 16, wherein
the non-pharmaceutical carrier provides for the form to be one of:
a rapidly dissolving tablet;
a rapidly dissolving hard candy;
a rapidly dissolving lollipop;
a chewable tablet; and
a jelly candy.
